(19) United States
(12) Patent Application Publication
(10) Pub. No.: US 2026/0224537 A1
(43) Pub. Date: Aug. 6, 2026

(54) METHODS AND COMPOSITIONS FOR TREATING GLUCOCORTICOID-MEDIATED DISEASES

(71) Applicant: SPARROW PHARMACEUTICALS, INC., Boston, MA (US)

(72) Inventor: David A. KATZ, Portland, OR (US)

(21) Appl. No.: 19/629,975

(22) Filed: Mar. 26, 2026

Related U.S. Application Data
(63) Continuation of application No. PCT/US2024/048659, filed on Sep. 26, 2024.
(60) Provisional application No. 63/585,618, filed on Sep. 27, 2023.

(51) Int. Cl. A61K 31/4196; A61K 31/573; A61P 19/00; A61P 37/06

(52) U.S. Cl. CPC A61K 31/4196; A61K 31/573; A61P 19/00; A61P 37/06

(57) ABSTRACT

The disclosure provides new and improved combination therapies using HSD-1 inhibitors and glucocorticoids in the treatment of glucocorticoid-mediated diseases.

This application is a continuation of International Patent Application No. PCT/US2024/048659, filed on Sep. 26, 2024, which claims the benefit of priority of U.S. Provisional Application No. 63/585,618, filed Sep. 27, 2023, the contents of each which are incorporated by reference as if written herein in their entirety.

Glucocorticoids (GCs) are corticosteroids that bind to the glucocorticoid receptor (GR), which is present in many cell types in the human body. GCs are involved in cardiovascular, metabolic, immunologic, osteal, muscular, dermatological, ocular, psychiatric, cognitive, circadian, and homeostatic functions. In addition, GCs can also bind to the mineralocorticoid receptor (MR) and non-genomic receptors.

Important natural GCs include cortisol (known medically as hydrocortisone) and corticosterone. Synthetic GCs include prednisolone, methylprednisolone, dexamethasone, and many others, as well as derivatives of these. Additionally, inactive congeners (e.g., cortisone, prednisone) that don't activate GR are commonly referred to as GCs. Both natural and synthetic GCs are used as medications to treat conditions that include autoimmune diseases and inflammatory conditions. However, an excess of either natural or synthetic GCs in humans can lead to myriad symptoms and illnesses. Such excesses are most commonly caused either by tumors that secrete cortisol or hormones that increase cortisol secretion (e.g., ACTH or CRH), or through excess administration of hydrocortisone or synthetic GCs during medical treatment.

11β-hydroxysteroid dehydrogenases (HSDs) are enzymes that regulate the intracellular levels of glucocorticoids. The HSD enzymes consist of two isoforms: the nicotinamide-adenine dinucleotide phosphate-dependent type 1 (HSD-1), which converts inactive cortisone to active cortisol, and the nicotinamide-adenine dinucleotide dependent oxidative type 2 (HSD-2), which converts cortisol to cortisone. HSD-1 is a major source of intracellular cortisol and is thought to be a major source of intracellular synthetic GC in many cell types. Excess intracellular GCs activate GR and MR, along with non-genomic receptors, resulting in the tissue-specific morbidity observed in patients with GC excess. Inhibition of HSD-1 may therefore ameliorate those symptoms.

Novel potent HSD-1 inhibitors, including 4-(5-(2-(4-chloro-2,6-difluorophenoxy)propan-2-yl)-4-methyl-4 h-1,2,4-triazol-3-yl)-3-fluorobenzamide and related compounds, have been described in U.S. Pat. No. 8,377,923, the contents of which are hereby incorporated by reference in their entirety, as inhibitors of HSD-1 with promising potential.

There exists a need for new and improved combination therapies using HSD-1 inhibitors and GCs in the treatment of GC-mediated diseases. Such combinations would be useful for treating the GC-mediated disease while ameliorating side effects from the GC itself.

Citation of any reference throughout this application is not to be construed as an admission that such reference is prior art to the present application.

### SUMMARY

Provided herein is a fixed dose combination comprising no more than about 6 mg of an HSD-1 inhibitor and between 1.5 and 150 mg of prednisolone.

Also provided is a fixed dose combination comprising no more than about 6 mg of an HSD-1 inhibitor and between about 1.5 and about 150 mg of prednisolone.

Also provided is a method of treatment of a prednisolone-mediated disease, comprising the administration of a fixed dose combination as recited herein to a patient in need thereof.

Also provided is a pharmaceutical composition comprising a pharmaceutically acceptable carrier and a fixed dose combination as recited herein.

### BRIEF DESCRIPTION OF THE DRAWINGS

FIG. 1 depicts the mean and confidence interval of the changes from baseline in C-reactive protein (CRP; medians and interquartile ranges) and erythrocyte sedimentation rate (ESR; means and confidence intervals of means) following 2 weeks of treatment with 10 mg prednisolone and placebo (PRL 10), 10 mg prednisolone and 6 mg clofutriben (PRL10 CLO6), 15 mg prednisolone and 6 mg clofutriben (PRL15 CLO6), 20 mg prednisolone and 6 mg clofutriben (PRL20 CLO6), and 30 mg prednisolone and 6 mg clofutriben (PRL30 CLO6) in clinical trial participants with polymyalgia rheumatica. The arrow indicates the direction of improvement within a given parameter.

FIG. 2 depicts the mean and confidence interval of the mean changes from baseline score change in the Health Assessment Questionnaire Disability Index (HAQ-DI) following 2 weeks of treatment with 10 mg prednisolone and placebo (PRL 10), 10 mg prednisolone and 6 mg clofutriben (PRL10 CLO6), 15 mg prednisolone and 6 mg clofutriben (PRL15 CLO6), 20 mg prednisolone and 6 mg clofutriben (PRL20 CLO6), and 30 mg prednisolone and 6 mg clofutriben (PRL30 CLO6) in clinical trial participants with polymyalgia rheumatica. The arrow indicates the direction of improvement.

FIG. 3 depicts the mean and confidence interval of the mean changes from baseline in B cell activating factor (BAFF) and CXC motif chemokine ligand 10 (CXCL10) following 2 weeks of treatment with 10 mg prednisolone and placebo (PRL 10), 10 mg prednisolone and 6 mg clofutriben (PRL10 CLO6), 15 mg prednisolone and 6 mg clofutriben (PRL15 CLO6), 20 mg prednisolone and 6 mg clofutriben (PRL20 CLO6), and 30 mg prednisolone and 6 mg clofutriben (PRL30 CLO6) in clinical trial participants with polymyalgia rheumatica.

FIG. 4 depicts the mean and confidence interval of the mean changes from baseline in IL-2 receptor subunit A (IL-2RA) and IL-6 following 2 weeks of treatment with 10 mg prednisolone and placebo (PRL 10), 10 mg prednisolone and 6 mg clofutriben (PRL10 CLO6), 15 mg prednisolone and 6 mg clofutriben (PRL15 CLO6), 20 mg prednisolone and 6 mg clofutriben (PRL20 CLO6), and 30 mg prednisolone and 6 mg clofutriben (PRL30 CLO6) in clinical trial participants with polymyalgia rheumatica.

FIG. 5 depicts the mean and confidence interval of the mean changes from baseline in granulocyte colony stimulating factor (G-CSF) and interferon gamma (IFNγ) following 2 weeks of treatment with 10 mg prednisolone and placebo (PRL 10), 10 mg prednisolone and 6 mg clofutriben (PRL10 CLO6), 15 mg prednisolone and 6 mg clofutriben (PRL15 CLO6), 20 mg prednisolone and 6 mg clofutriben (PRL20 CLO6), and 30 mg prednisolone and 6 mg clofutriben (PRL30 CLO6) in clinical trial participants with polymyalgia rheumatica.

FIG. 6 depicts the mean and confidence interval of the mean changes from baseline in IL-1 receptor antagonist (IL-1RA) and TNF alpha following 2 weeks of treatment with 10 mg prednisolone and placebo (PRL 10), 10 mg prednisolone and 6 mg clofutriben (PRL10 CLO6), 15 mg prednisolone and 6 mg clofutriben (PRL15 CLO6), 20 mg prednisolone and 6 mg clofutriben (PRL20 CLO6), and 30 mg prednisolone and 6 mg clofutriben (PRL30 CLO6) in clinical trial participants with polymyalgia rheumatica.

FIG. 7 depicts mean and confidence interval of the mean changes from baseline in circulating B cell and IL-6R+ cell counts following 2 weeks of treatment with 10 mg prednisolone and placebo (PRL 10), 10 mg prednisolone and 6 mg clofutriben (PRL10 CLO6), 15 mg prednisolone and 6 mg clofutriben (PRL15 CLO6), 20 mg prednisolone and 6 mg clofutriben (PRL20 CLO6), and 30 mg prednisolone and 6 mg clofutriben (PRL30 CLO6) in clinical trial participants with polymyalgia rheumatica.

FIG. 8 depicts mean and confidence interval of the mean changes from baseline in IL-17A+ cell and memory B cell counts following 2 weeks of treatment with 10 mg prednisolone and placebo (PRL 10), 10 mg prednisolone and 6 mg clofutriben (PRL10 CLO6), 15 mg prednisolone and 6 mg clofutriben (PRL15 CLO6), 20 mg prednisolone and 6 mg clofutriben (PRL20 CLO6), and 30 mg prednisolone and 6 mg clofutriben (PRL30 CLO6) in clinical trial participants with polymyalgia rheumatica.

FIG. 9 depicts mean and confidence interval of the mean changes from baseline in T(CD3+) cell and Treg cell counts following 2 weeks of treatment with 10 mg prednisolone and placebo (PRL 10), 10 mg prednisolone and 6 mg clofutriben (PRL10 CLO6), 15 mg prednisolone and 6 mg clofutriben (PRL15 CLO6), 20 mg prednisolone and 6 mg clofutriben (PRL20 CLO6), and 30 mg prednisolone and 6 mg clofutriben (PRL30 CLO6) in clinical trial participants with polymyalgia rheumatica.

FIG. 10 depicts mean and confidence interval of the mean changes from baseline in fibrinogen following 2 weeks of treatment with 10 mg prednisolone and placebo (PRL 10), 10 mg prednisolone and 6 mg clofutriben (PRL10 CLO6), 15 mg prednisolone and 6 mg clofutriben (PRL15 CLO6), 20 mg prednisolone and 6 mg clofutriben (PRL20 CLO6), and 30 mg prednisolone and 6 mg clofutriben (PRL30 CLO6) in clinical trial participants with polymyalgia rheumatica. The arrow indicates the direction of improvement.

FIG. 11 depicts the mean and confidence interval of the mean changes from baseline in monocyte, neutrophil, and platelet counts following 2 weeks of treatment with 10 mg prednisolone and placebo (PRL 10), 10 mg prednisolone and 6 mg clofutriben (PRL10 CLO6), 15 mg prednisolone and 6 mg clofutriben (PRL15 CLO6), 20 mg prednisolone and 6 mg clofutriben (PRL20 CLO6), and 30 mg prednisolone and 6 mg clofutriben (PRL30 CLO6) in clinical trial participants with polymyalgia rheumatica.

FIG. 12 depicts the mean and confidence interval of the mean changes from baseline in bone formation biomarkers OC, P1NP, and BSAP following 2 weeks of treatment with 10 mg prednisolone and placebo (PRL 10), 10 mg prednisolone and 6 mg clofutriben (PRL10 CLO6), 15 mg prednisolone and 6 mg clofutriben (PRL15 CLO6), 20 mg prednisolone and 6 mg clofutriben (PRL20 CLO6), and 30 mg prednisolone and 6 mg clofutriben (PRL30 CLO6) in clinical trial participants with polymyalgia rheumatica.

FIG. 13 depicts the mean and confidence interval of the mean changes from baseline in bone resorption biomarker CTX following 2 weeks of treatment with 10 mg prednisolone and placebo (PRL 10), 10 mg prednisolone and 6 mg clofutriben (PRL10 CLO6), 15 mg prednisolone and 6 mg clofutriben (PRL15 CLO6), 20 mg prednisolone and 6 mg clofutriben (PRL20 CLO6), and 30 mg prednisolone and 6 mg clofutriben (PRL30 CLO6) in clinical trial participants with polymyalgia rheumatica.

FIG. 14 depicts the mean and confidence interval of the mean changes from baseline in LDL cholesterol following 2 weeks of treatment with 10 mg prednisolone and placebo (PRL 10), 10 mg prednisolone and 6 mg clofutriben (PRL10 CLO6), 15 mg prednisolone and 6 mg clofutriben (PRL15 CLO6), 20 mg prednisolone and 6 mg clofutriben (PRL20 CLO6), and 30 mg prednisolone and 6 mg clofutriben (PRL30 CLO6).

FIG. 15 depicts the mean and confidence interval of the mean changes from baseline in HDL cholesterol following 2 weeks of treatment with 10 mg prednisolone and placebo (PRL 10), 10 mg prednisolone and 6 mg clofutriben (PRL10 CLO6), 15 mg prednisolone and 6 mg clofutriben (PRL15 CLO6), 20 mg prednisolone and 6 mg clofutriben (PRL20 CLO6), and 30 mg prednisolone and 6 mg clofutriben (PRL30 CLO6).

FIG. 16 depicts the mean and confidence interval of the mean changes from baseline in OGTT glucose AUC and HbA1c and the medians and interquartile ranges of HOMA-IR following 2 weeks of treatment with 10 mg prednisolone and placebo (PRL 10), 10 mg prednisolone and 6 mg clofutriben (PRL10 CLO6), 15 mg prednisolone and 6 mg clofutriben (PRL15 CLO6), 20 mg prednisolone and 6 mg clofutriben (PRL20 CLO6), and 30 mg prednisolone and 6 mg clofutriben (PRL30 CLO6).

FIG. 17 depicts the mean and confidence interval of the mean changes from baseline in coagulation parameters Prothrombin International Normalized Ratio (INR), and Prothrombin Time (PT) following 2 weeks of treatment with 10 mg prednisolone and placebo (PRL 10), 10 mg prednisolone and 6 mg clofutriben (PRL10 CLO6), 15 mg prednisolone and 6 mg clofutriben (PRL15 CLO6), 20 mg prednisolone and 6 mg clofutriben (PRL20 CLO6), and 30 mg prednisolone and 6 mg clofutriben (PRL30 CLO6).

FIG. 18 depicts the urinary ratio (tetrahydrocortisol+allotetrahydrocortisol)/tetrahydrocortisone from morning spot urine samples collected at Baseline (BL), after 2 weeks of prednisolone 10 mg treatment (PRL), after 2 weeks of prednisolone 10-30 mg+clofutriben 6 mg treatment (PRL CLO6), and after 4 weeks washout following the last clofutriben dose (WO) from clinical trial participants with polymyalgia rheumatica.

FIG. 19 depicts the mean and confidence interval of the mean changes from baseline in cholesterol following 2 weeks of treatment with 10 mg prednisolone and placebo (PRL 10), 10 mg prednisolone and 6 mg clofutriben (PRL10 CLO6), 15 mg prednisolone and 6 mg clofutriben (PRL15 CLO6), 20 mg prednisolone and 6 mg clofutriben (PRL20 CLO6), and 30 mg prednisolone and 6 mg clofutriben (PRL30 CLO6). The arrow shows the direction of improvement.

FIG. 20 depicts the mean and confidence interval of the mean changes from baseline (right image) in triglycerides following 2 weeks of treatment with 10 mg prednisolone and placebo (PRL 10), 10 mg prednisolone and 6 mg clofutriben (PRL10 CLO6), 15 mg prednisolone and 6 mg clofutriben (PRL15 CLO6), 20 mg prednisolone and 6 mg clofutriben (PRL20 CLO6), and 30 mg prednisolone and 6 mg clofutriben (PRL30 CLO6). The arrow shows the direction of improvement.

FIG. 21 depicts the mean and confidence interval of the mean changes from baseline (right image) on Activated partial thromboplastin time (APTT) following 2 weeks of treatment with 10 mg prednisolone and placebo (PRL 10), 10 mg prednisolone and 6 mg clofutriben (PRL10 CLO6), 15 mg prednisolone and 6 mg clofutriben (PRL15 CLO6), 20 mg prednisolone and 6 mg clofutriben (PRL20 CLO6), and 30 mg prednisolone and 6 mg clofutriben (PRL30 CLO6). The arrow shows the direction of improvement.

FIG. 22 depicts the mean and confidence interval of the mean changes from baseline with regard to ECG heart rate following 2 weeks of treatment with 10 mg prednisolone and placebo (PRL 10), 10 mg prednisolone and 6 mg clofutriben (PRL10 CLO6), 15 mg prednisolone and 6 mg clofutriben (PRL15 CLO6), 20 mg prednisolone and 6 mg clofutriben (PRL20 CLO6), and 30 mg prednisolone and 6 mg clofutriben (PRL30 CLO6).

FIG. 23 depicts the mean and confidence interval of the mean changes from baseline with regard to pain intensity, stiffness intensity, fatigue intensity, and pain chronicity following 2 weeks of treatment with 10 mg prednisolone and placebo (PRL 10), 10 mg prednisolone and 6 mg clofutriben (PRL10 CLO6), 15 mg prednisolone and 6 mg clofutriben (PRL15 CLO6), 20 mg prednisolone and 6 mg clofutriben (PRL20 CLO6), and 30 mg prednisolone and 6 mg clofutriben (PRL30 CLO6).

FIG. 24 depicts the distribution of data with regard to the reference range (top two images) and the mean and confidence interval of the mean changes from baseline (bottom two images) on plasma adrenocorticotropic hormone (plasma ACTH) and serum cortisol following 2 weeks of treatment with 10 mg prednisolone and placebo (PRL 10), 10 mg prednisolone and 6 mg clofutriben (PRL10 CLO6), 15 mg prednisolone and 6 mg clofutriben (PRL15 CLO6), 20 mg prednisolone and 6 mg clofutriben (PRL20 CLO6), and 30 mg prednisolone and 6 mg clofutriben (PRL30 CLO6). The dashed green line represents the upper limit of the laboratory's reference range. The red and yellow lines represent thresholds of risk for adrenal insufficiency from a joint European Society for Endocrinology/Endocrine Society guideline.

FIG. 25 depicts the mean and confidence interval of the mean changes from baseline of serum cortisone and aldosterone following 2 weeks of treatment with 10 mg prednisolone and placebo (PRL 10), 10 mg prednisolone and 6 mg clofutriben (PRL10 CLO6), 15 mg prednisolone and 6 mg clofutriben (PRL15 CLO6), 20 mg prednisolone and 6 mg clofutriben (PRL20 CLO6), and 30 mg prednisolone and 6 mg clofutriben (PRL30 CLO6).

FIG. 26 depicts the mean and confidence interval of the mean changes from baseline on dehydroepiandrosterone sulfate (DHEA-S) and testosterone following 2 weeks of treatment with 10 mg prednisolone and placebo (PRL 10), 10 mg prednisolone and 6 mg clofutriben (PRL10 CLO6), 15 mg prednisolone and 6 mg clofutriben (PRL15 CLO6), 20 mg prednisolone and 6 mg clofutriben (PRL20 CLO6), and 30 mg prednisolone and 6 mg clofutriben (PRL30 CLO6).

### DETAILED DESCRIPTION OF THE DISCLOSURE

Provided herein is a fixed dose combination comprising no more than about 6 mg of an HSD-1 inhibitor and between 1.5 and 150 mg of prednisolone.

Also provided is a fixed dose combination comprising no more than about 6 mg of an HSD-1 inhibitor and between about 1.5 and about 150 mg of prednisolone.

In some embodiments, the HSD-1 inhibitor is a pseudo-irreversible inhibitor.

In some embodiments, the HSD-1 inhibitor is SPI-62.

In some embodiments, the fixed dose combination comprises no more than about 3 mg of SPI-62 and between about 1.5 and 7.5 mg of prednisolone.

In some embodiments, the fixed dose combination comprises no more than about 3 mg of SPI-62 and between about 1.5 and about 7.5 mg of prednisolone.

In some embodiments, the fixed dose combination comprises about 1.2 mg of SPI-62.

In some embodiments, the fixed dose combination comprises between about 1.5 and about 3 mg of prednisolone.

In some embodiments, the fixed dose combination comprises between about 2 and about 3 mg of prednisolone.

In some embodiments, the fixed dose combination has a therapeutic effect corresponding to a dose of about 1 mg of prednisolone administered alone.

In some embodiments, the fixed dose combination comprises about 3 mg of SPI-62.

In some embodiments, the fixed dose combination comprises between about 3.75 and about 7.5 mg of prednisolone.

In some embodiments, the fixed dose combination comprises between about 5 and about 7.5 mg of prednisolone.

In some embodiments, the combined dosage is sufficient to achieve a therapeutic effect corresponding to a dose of about 2.5 mg of prednisolone administered alone.

In some embodiments, the fixed dose combination comprises about 6 mg of SPI-62 and between about 5 and about 150 mg of prednisolone.

In some embodiments, the fixed dose combination comprises between about 7.5 and about 15 mg of prednisolone.

In some embodiments, the fixed dose combination comprises between about 10 and about 15 mg of prednisolone.

In some embodiments, the combined dosage is sufficient to achieve a therapeutic effect corresponding to a dose of about 5 mg of prednisolone administered alone.

In some embodiments, the fixed dose combination comprises between about 15 and about 30 mg of prednisolone.

In some embodiments, the fixed dose combination comprises between about 20 and about 30 mg of prednisolone.

In some embodiments, the combined dosage is sufficient to achieve a therapeutic effect corresponding to a dose of about 10 mg of prednisolone administered alone.

In some embodiments, the fixed dose combination comprises between about 30 and about 60 mg of prednisolone.

In some embodiments, the fixed dose combination comprises between about 40 and about 60 mg of prednisolone.

In some embodiments, the combined dosage is sufficient to achieve sufficient to achieve a therapeutic effect corresponding to a dose of about 20 mg of prednisolone administered alone.

In some embodiments, the fixed dose combination comprises between about 75 and about 150 mg of prednisolone.

In some embodiments, the fixed dose combination comprises between about 100 and about 150 mg of prednisolone.

In some embodiments, the combined dosage is sufficient to achieve a therapeutic effect corresponding to a dose of about 50 mg of prednisolone administered alone.

In some embodiments, the SPI-62 and the prednisolone are administered separately.

In some embodiments, the SPI-62 and the prednisolone are co-formulated.

In some embodiments, the SPI-62 and prednisolone are both formulated for immediate release.

In some embodiments, the fixed dose composition exhibits lowered toxicity than prednisolone administered alone.

In some embodiments, the toxicity is glucocorticoid-induced osteoporosis (GIOP).

In some embodiments, the lowered toxicity is characterized by increased osteocalcin, increased procollagen type 1 N-terminal propeptide, increased bone-specific alkaline phosphatase, or decreased C-terminal telopeptide of type 1 collagen.

In some embodiments, the toxicity is bradycardia.

In some embodiments, the lowered toxicity is characterized by increased heart rate.

Also provided is a method of treatment of a prednisolone-mediated disease, comprising the administration of a fixed dose combination as recited herein to a patient in need thereof.

In some embodiments, the prednisolone-mediated disease is chosen from an autoimmune disease, an inflammatory condition, an allergic condition, a dermatologic disease, an endocrine condition, a gastrointestinal disease, a hematologic disease, a neoplastic condition, a nervous system condition, an ophthalmic condition, a pulmonary disease, a renal condition, a rheumatologic condition, and an infectious disease.

In some embodiments, the prednisolone-mediated disease is an autoimmune disease.

In some embodiments, the autoimmune disease is polymyalgia rheumatica.

In some embodiments, the prednisolone-mediated disease is an inflammatory condition.

In some embodiments, the prednisolone-mediated disease is chosen from:
an allergic condition chosen from atopic dermatitis, drug hypersensitivity, seasonal or perennial allergic rhinitis, and serum sickness;
a dermatologic disease chosen from bullous dermatitis herpetiformis, contact dermatitis, exfoliative erythroderma, mycosis fungoides, pemphigus, and severe erythema multiforme (Stevens-Johnson syndrome);
an endocrine condition chosen from congenital adrenal hyperplasia, hypercalcemia of malignancy, nonsuppurative thyroiditis, and primary or secondary adrenocortical insufficiency;
a gastrointestinal disease chosen from Crohn's disease and ulcerative colitis;
a hematologic disease chosen from acquired (autoimmune) hemolytic anemia, Diamond-Blackfan anemia, adult idiopathic thrombocytopenic purpura, pure red cell aplasia, and adult secondary thrombocytopenia;
a neoplastic condition chosen from acute leukemia and aggressive lymphomas;
a nervous system condition chosen from multiple sclerosis and cerebral edema associated with a primary or metastatic brain tumor, craniotomy, or head injury;
an ophthalmic condition chosen from sympathetic ophthalmia, uveitis, and ocular inflammatory conditions unresponsive to topical corticosteroids;
a pulmonary disease chosen from chronic obstructive pulmonary disease (COPD), allergic bronchopulmonary aspergillosis, aspiration pneumonitis, asthma, fulminating or disseminated pulmonary tuberculosis, hypersensitivity pneumonitis, idiopathic bronchiolitis obliterans, idiopathic eosinophilic pneumonias, idiopathic pulmonary fibrosis, Pneumocystis carinii pneumonia (PCP), and symptomatic sarcoidosis;
a renal condition chosen from nephrotic syndrome;
a rheumatologic condition chosen from acute gouty arthritis, ankylosing spondylitis, dermatomyositis, polymyositis, polymyalgia rheumatica, temporal arteritis, psoriatic arthritis, relapsing polychondritis, rheumatoid arthritis, juvenile rheumatoid arthritis, Sjogren's syndrome, systemic lupus erythematosus, and vasculitis; and
an infectious disease chosen from trichinosis and tuberculous meningitis.

In some embodiments, the administration results in an increase in bone formation or a decrease in bone resorption in the patient.

In some embodiments, the increased bone formation is characterized by increased osteocalcin, increased procollagen type 1 N-terminal propeptide, increased bone-specific alkaline phosphatase, or the decreased bone resorption is characterized by decreased C-terminal telopeptide of type 1 collagen.

Also provided is a method of treatment of adrenal insufficiency or glucocorticoid withdrawal syndrome, comprising the administration of a fixed dose combination disclosed herein to a patient in need thereof.

In some embodiments, the glucocorticoid is prednisolone.

Also provided is a pharmaceutical composition comprising a pharmaceutically acceptable carrier and a fixed dose combination as recited herein.

In some embodiments, the pharmaceutical composition is formulated for oral administration

### Pseudo-Irreversible HSD-1 Inhibitor Compound Structures

A number of pseudo-irreversible HSD-1 inhibitors are known and available in the art, including, but not limited to, SPI-62, ABT-384, MK-0736, MK-0916, BMS-823778, UE-2343, AMG-221, KR-67607, BI-187004, SAR-184481, Compound A, Compound B, Compound C, Compound D, BMS-823778, or a pharmaceutically acceptable salt thereof. As would be understood by one of skill in the art, any pseudo-irreversible HSD-1 inhibitor described herein or known or available in the art may be used as described herein and is encompassed within the scope of the present disclosure.

The structures of some pseudo-irreversible HSD-1 inhibitors described herein are set forth below. All structures and IUPAC names were generated using ChemDraw 20.0.

As used herein, “SPI-62” and “clofutriben” refer to 4-(5-(2-(4-chloro-2,6-difluorophenoxy)propan-2-yl)-4-methyl-4H-1,2,4-triazol-3-yl)-3-fluorobenzamide.

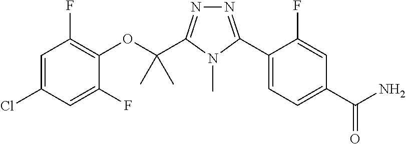 [SMILES: Cn1c(-c2ccc(C(N)=O)cc2F)nnc1C(C)(C)Oc1c(F)cc(Cl)cc1F]

As used herein, “BMS-823778” refers to 2-(3-(1-(4-chlorophenyl)cyclopropyl)-[1,2,4]triazolo[4,3-a]pyridin-8-yl)propan-2-ol.

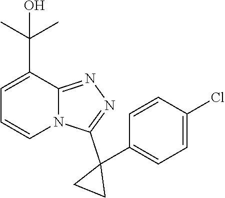 [SMILES: CC(C)(O)c1cccn2c(C3(c4ccc(Cl)cc4)CC3)nnc12]

As used herein, “ABT-384” refers to (1s,3R,4r,5S,7s)-4-(2-methyl-2-(4-(5-(trifluoromethyl)pyridin-2-yl)piperazin-1-yl)propanamido)adamantane-1-carboxamide.

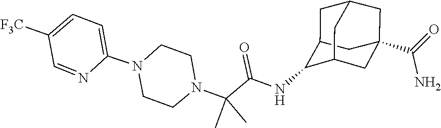 [SMILES: CC(C)(C(=O)N[C@H]1C2CC3CC1C[C@@](C(N)=O)(C3)C2)N1CCN(c2ccc(C(F)(F)F)cn2)CC1]

As used herein, “UE-2343” refers to (5-(1H-pyrazol-4-yl)thiophen-3-yl)((1R,3r,5S)-3-hydroxy-3-(pyrimidin-2-yl)-8-azabicyclo[3.2.1]octan-8-yl)methanone.

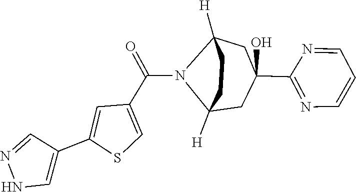

As used herein, “MK-0736” refers to 3-(4-(3-(ethylsulfonyl)propyl)bicyclo[2.2.2]octan-1-yl)-4-methyl-5-(2-(trifluoromethyl)phenyl)-4H-1,2,4-triazole.

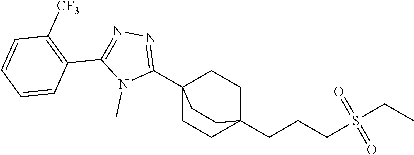 [SMILES: CCS(=O)(=O)CCCC12CCC(c3nnc(-c4ccccc4C(F)(F)F)n3C)(CC1)CC2]

As used herein, “AMG-221” refers to (S,E)-2-(((1S,2S,4R)-bicyclo[2.2.1]heptan-2-yl)imino)-5-isopropyl-5-methylthiazolidin-4-one.

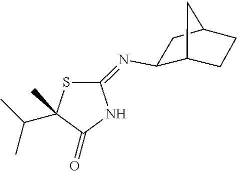 [SMILES: CC(C)[C@]1(C)S/C(=N/C2CC3CCC2C3)NC1=O]

As used herein, “MK-0916” refers to 3-((1s,3s)-1-(4-chlorophenyl)-3-fluorocyclobutyl)-4,5-dicyclopropyl-4H-1,2,4-triazole.

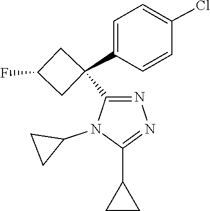 [SMILES: F[C@H]1C[C@@](c2ccc(Cl)cc2)(c2nnc(C3CC3)n2C2CC2)C1]

As used herein, “KR-67607” refers to (1s,3R,4s,5S,7s)-4-(2-(6-(2,6-dichloro-4-(trifluoromethyl)phenyl)-4-methyl-1,1-dioxido-1,2,6-thiadiazinan-2-yl)acetamido)adamantane-1-carboxamide.

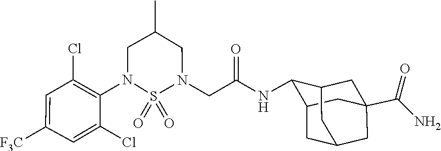 [SMILES: CC1CN(CC(=O)NC2C3CC4CC2CC(C(N)=O)(C4)C3)S(=O)(=O)N(c2c(Cl)cc(C(F)(F)F)cc2Cl)C1]

As used herein, “BI-187004” refers to (4aR,9aS)-1-(1H-benzo[d]imidazole-6-carbonyl)-2,3,4,4a,9,9a-hexahydro-1H-indeno[2,1-b]pyridine-6-carbonitrile.

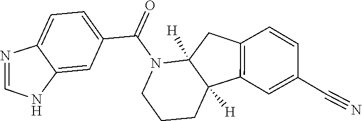 [SMILES: N#Cc1ccc2c(c1)[C@H]1CCCN(C(=O)c3ccc4nc[nH]c4c3)[C@H]1C2]

As used herein, “SAR-184841” refers to 4-(5-(4-(tert-butyl)piperazin-1-yl)pyridin-2-yl)-N-((1R,3S,5s,7s)-5-carbamoyladamantan-2-yl)-3,4-dihydroquinoxaline-1(2H)-carboxamide.

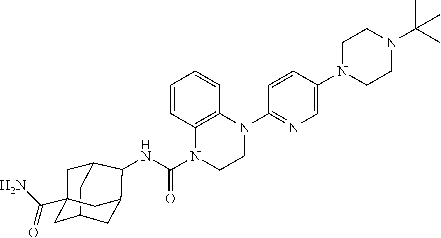 [SMILES: CC(C)(C)N1CCN(c2ccc(N3CCN(C(=O)NC4C5CC6CC4CC(C(N)=O)(C6)C5)c4ccccc43)nc2)CC1]

As used herein, “Compound A” refers to (1s,3R,5S,7s)-4-(2-(4-methoxyphenoxy)-2-methylpropanamido)adamantane-1-carboxamide.

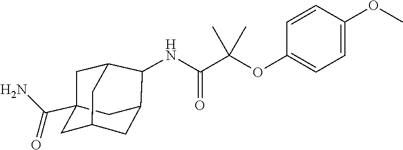 [SMILES: COc1ccc(OC(C)(C)C(=O)NC2C3CC4CC2CC(C(N)=O)(C4)C3)cc1]

As used herein, “Compound B” refers to 3,4-dihydroquinolin-1(2H)-yl[4-(1H-imidazol-5-yl)piperidin-1-yl]methanone.

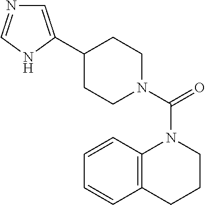 [SMILES: O=C(N1CCC(c2cnc[nH]2)CC1)N1CCCc2ccccc21]

As used herein, “Compound C” refers to (5R)-2-[(2-fluorophenyl)amino]-5-(1-methylethyl)-1,3-thiazol-4(5H)-one.

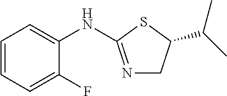 [SMILES: CC(C)[C@@H]1CN=C(Nc2ccccc2F)S1]

As used herein, “Compound D” refers to 3-(2-fluoroethyl)-4-((4-[(2S)-1,1,1-trifluoro-2-hydroxypropan-2-yl]phenyl)sulfonyl)benzonitrile.

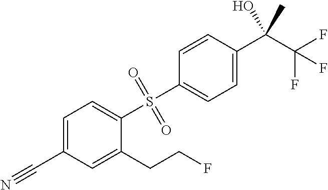 [SMILES: C[C@](O)(c1ccc(S(=O)(=O)c2ccc(C#N)cc2CCF)cc1)C(F)(F)F]

In some embodiments, a pseudo-irreversible HSD-1 inhibitor is administered in a single dose of from about 1 to about 6 mg, including 1 mg, 2 mg, 3 mg, 4 mg, 5 mg, 6 mg, or the like. In some embodiments, a sub-milligram dose may be any dose about equal to, or less than, 1 mg, such as 0.05 mg, 0.1 mg, 0.15 mg, 0.2 mg, 0.25 mg, 0.3 mg, 0.35 mg, 0.4 mg, 0.45 mg, 0.5 mg, 0.55 mg, 0.6 mg, 0.65 mg, 0.7 mg, 0.75 mg, 0.8 mg, 0.85 mg, 0.9 mg, 0.95 mg, or 1 mg.

In some embodiments, a pseudo-irreversible HSD-1 inhibitor may be administered in multiple doses for a specified period of time. Multiple doses may refer to single doses per day or week for a period of time deemed appropriate by a clinician or practitioner.

In some embodiments, a period of time for administration of a pseudo-irreversible HSD-1 inhibitor may be any period of time, such as including, but not limited to, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, or the like. A period of time in accordance with the present disclosure may be, any period of time, for example, a period of 1-14 days, or 7-14 days, or 7-10 days, or 10-14 days. In other embodiments, a period of time for administration of an HSD-1 inhibitor may be from 7-30 days, or 7-21 days, or the like. In some embodiments, the period of time for administration of a pseudo-irreversible HSD-1 inhibitor may be 1 day, 6 days, or 12 days. In some embodiments, a pseudo-irreversible HSD-1 inhibitor may be administered to a patient on a continual basis. In some embodiments, administration of a dosage of a pseudo-irreversible HSD-1 inhibitor may be for 1 month, or 2 months, or 3 months, or 4 months, or 5 months, or 6 months, or 7 months, or 8 months, or 9 months, or 10 months, or 11 months, or 12 months, or 15 months, or 18 months, or 24 months, or 36 months, or 48 months, or longer.

In some embodiments, an initial dose of a pseudo-irreversible HSD-1 inhibitor initially binds to, and remains bound to, its target in tissues. In some embodiments, up to about 3 mg of the pseudo-irreversible HSD-1 inhibitor is sequestered from central circulation. In some embodiments, the pseudo-irreversible HSD-1 inhibitor exhibits nonlinear pharmacokinetics after a single dose in the sub-milligram to low-milligram range, with dose-proportional exposures after multiple doses. In some embodiments, plasma exposures of the HSD-1 inhibitor are less than dose-proportional after low single doses and dose-proportional after multiple low doses. In some embodiments, the doses are 6 mg or lower, e.g., 6 mg, or 5 mg, or 4 mg, or 3 mg, or 2 mg, or 1 mg, or less than 1 mg. In some embodiments, the doses are 3 mg or lower.

### Definitions

The definitions and methods provided define the present disclosure and guide those of ordinary skill in the art in the practice of the present disclosure. Unless otherwise noted, terms are to be understood according to conventional usage by those of ordinary skill in the relevant art.

As used herein, a “fixed dose combination” refers to a combination of two active pharmaceutical ingredients in defined doses. In some embodiments, the fixed dose combination is presented in a single dosage unit (e.g., a tablet or a capsule). In some embodiments, the fixed dose combination is presented as a combination of two drugs or active ingredients administered as two distinct dosage units.

As used herein, a “pseudo-irreversible inhibitor” refers to a compound that binds to, interacts with, elicits a conformational change, or otherwise inhibits or abolishes the activity of HSD-1 and possesses fast-on, slow-off binding kinetics to HSD-1 or can form a tertiary complex with HSD-1 and NADPH in the active site.

As used herein, the terms “subject” or “patient” refer to a human. Unless otherwise noted, the terms “patient” or “subject” are used herein interchangeably.

As used herein, “treating,” “treatment,” and the like means the administration of therapy to an individual who already manifests at least one symptom of a disease or condition or who has previously manifested at least one symptom of a disease or condition. For example, “treating” can include alleviating, abating, or ameliorating a disease or condition symptoms, preventing additional symptoms, ameliorating the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition. For example, the term “treating” in reference to a disorder means a reduction in severity of one or more symptoms associated with that particular disorder. Therefore, treating a disorder does not necessarily mean a reduction in severity of all symptoms associated with a disorder and does not necessarily mean a complete reduction in the severity of one or more symptoms associated with a disorder. As related to the present disclosure, the term may also mean the administration of pharmacological substances or formulations, or the performance of non-pharmacological methods including, but not limited to, radiation therapy and surgery.

The term “prednisolone-mediated disease”, as used herein, refers to a disease or condition which is amenable to treatment with prednisolone.

The term “about,” as used herein, is intended to qualify the numerical values which it modifies, denoting such a value as variable within a range. When no particular range, such as a margin of error or a standard deviation to a mean value given in a chart or table of data, is recited, the term “about” should be understood to mean the greater of the range which would encompass the recited value, the range which would be included by rounding up or down to that figure as well taking into account significant figures, and the range which would encompass the recited value plus or minus 20%.

The terms “a,” and “an,” and “the,” and similar references used in the context of describing a particular embodiment (especially in the context of certain of the following claims) can be construed to cover both the singular and the plural, unless specifically noted otherwise. The term “or” as used herein, including the claims, is used inclusively unless explicitly indicated to refer to alternatives only or the alternatives are mutually exclusive.

The term “corresponding”, as used herein, refers to when a fixed dose combination has a therapeutic effect that is equivalent, non-inferior, or superior to that of another pharmaceutical ingredient or ingredients with the same therapeutic target or effects.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

Embodiments of the present disclosure are provided in the following examples. These examples are presented only by way of illustration and to assist one of ordinary skill in using the disclosure. The examples are not intended in any way to otherwise limit the scope of the disclosure.

### Example 1. Clinical Trial Results

In an ongoing clinical trial, patients with polymyalgia rheumatica (PMR), who receive prednisolone 10 mg daily for at least 7 days prior to Day 1, are in remission or have low disease activity, and who meet the inclusion and exclusion criteria (Table 1), enter a 4-week treatment period.

TABLE 1
Inclusion/Exclusion criteria

| Inclusion Criteria | Exclusion Criteria |
|---|---|
| 1. Able to understand the nature and scope | 1. Known hypersensitivity to prednisolone, |
| of the trial. | SPI-62 or any of the excipients. |
| 2. Written informed consent given prior to | 2. Systemic fungal infection. |
| any trial-related procedures. | 3. Administration of any vaccine within 4 |
| 3. At least 18 years old. | weeks prior to Day 1 or during the trial. |
| 4. Diagnosis of polymyalgia rheumatica | 4. Any relative contraindication for |
| according to EULAR/ACR | prednisolone of sufficient severity that |
| classification criteria confirmed by a | the investigator considers the potential |
| rheumatologist. | participant should receive immediate |
| 5. Absence of PMR relapse based on | additional treatment or not continue |
| symptoms. | prednisolone. |
| The Investigator does not | 5. Fulfills all criteria and clearly qualifies |
| consider either prednisolone | to use methotrexate according to |
| dose increase or additional | national or EULAR/ACR guideline for |
| treatment necessary based on | treatment of PMR, unless the potential |
| PMR symptoms from the time | participant refuses to take methotrexate |
| of participant consent through | or has a contraindication to |
| the Baseline Visit, inclusive. | methotrexate. |
| 6. Absence of PMR relapse based on acute | 6. A diagnosis or any clinical features of |
| phase markers: | GCA. |
| At Screening, ESR ≤40 mm/h. | 7. Any known autoimmune disease (e.g., |
| If a Day-1 ESR value is >40 | late-onset rheumatoid arthritis) other |
| mm/h and >50% higher than | than PMR. |
| the Screening value, the | 8. Use of medications other than oral |
| potential participant will not be | prednisolone for treatment of PMR. |
| eligible. | 9. Use of other medications likely to |
| 7. Daily oral prednisolone 10 mg dose that | interfere with trial assessments. |
| will have been stable for at least 1 week | 10. History of clinically significant labile |
| at the Baseline Visit and is expected to | diabetes or hypertension during |
| remain stable during the treatment | glucocorticoid therapy. |
| period. | 11. History or diagnosis of endogenous |
| | hypercortisolism. |
| | 12. Moderate or severe renal impairment. |
| | 13. Medically significant liver disease. |
| | 14. Medically significant cardiovascular or |
| | ECG abnormalities. |
| | 15. History of idiopathic thrombocytopeni |
| | purpura. |
| | 16. Recent SARS-CoV-2 infection. |
| | 17. History of cancer within 3 years other |
| | than non-melanoma skin cancer or |
| | early-stage prostate cancer. |
| | 18. Any major surgery, or significant post- |
| | operative sequelae, within 1 month prior |
| | to informed consent or planned during |
| | the trial. |
| | 19. Pregnant, lactating, or woman of child- |
| | bearing potential unwilling to adhere to |
| | highly effective contraceptive use or |
| | abstinence. |
| | 20. Man unwilling to adhere to highly |
| | effective contraceptive use or |
| | abstinence. |
| | 21. Participation in any clinical trial of an |
| | experimental drug within 5 half-lives or |
| | 1 month prior to informed consent. |
| | 22. Receipt of blood products within 2 |
| | months prior to Screening. |
| | 23. Donation of blood from 3 months prior |
| | to Screening, plasma from 2 weeks prior |
| | to Screening, or platelets from 6 weeks |
| | prior to Screening . . . |
| | 24. Any current or prior medical condition |
| | expected to interfere with the conduct of |
| | the trial or the evaluation of its results, |
| | or that requires immediate medical |
| | attention. |
| | 25. Employees of the Sponsor or employees |
| | or relatives of the Investigator. |
| | 26. Persons committed to an institution by |
| | virtue of a judicial order or an order |
| | issued by an administrative authority. |

EULAR/ACR = European League Against Rheumatism/American College of Rheumatology
PMR = polymyalgia rheumatica
ESR = erythrocyte sedimentation rate
GCA = giant cell arteritis
SARS-CoV-2 = severe acute respiratory coronavirus type 2

From Day 1 to Week 2, participants continue to receive prednisolone 10 mg and placebo for SPI-62, each daily. From Week 2 to Week 4, participants receive prednisolone, at a dose or randomized doses assigned per cohort, and SPI-62 6 mg, each daily. The order of treatments are not randomized due to the long pharmacological half-life of SPI-62, which is understood to be approximately 4-6 weeks based on data collected up to 2-2.5 weeks after cessation of SPI-62 administration in prior clinical trials. Participants are blinded to the order of treatments. Participants are enrolled in cohorts of approximately 12.
During Cohort 1, 12 participants received prednisolone 10 mg with SPI-62 6 mg. Two participants were erroneously dosed with SPI-62 on Day 1. Hence, only 10 participants also received prednisolone 10 mg with placebo for SPI-62. Two participants' prednisolone dose was increased to 15 mg prior to Week 4 following adverse events of PMVR aggravated. Those two participants' data are represented in the Figures in the PRL15 CLO6 regimen. Two additional participants showed no prednisolone levels in Week 4, which is suggestive that they received prednisone. Those two participants' data are represented in the Figures only in the PRL10 regimen.
During Cohort 2, 12 participants received prednisolone 15 mg (50% dose adjustment) with SPI-62 6 mg. All also received prednisolone 10 mg with placebo for SPI-62.
During Cohort 3, 11 participants received prednisolone 20 mg (100% dose adjustment from Cohort 1) with clofutriben (i.e., SPI-62) 6 mg. All also received prednisolone 10 mg with placebo for SPI-62. One additional participant showed prednisolone and prednisone levels at Week 4 consistent with intake of prednisolone 10 mg and prednisone 10 mg. That participant's data are represented in the Figures in the PRL10 and PRL10 CLO6 regimens.
During Cohort 4, 11 participants received prednisolone 30 mg (100% dose adjustment from Cohort 2) with clofutriben (i.e., SPI-62) 6 mg.
Two additional participants dropped out of the trial before they received clofutriben. Those participants' data are represented in the Figures in the PRL10 regimen.

Interim results of Cohorts 1 & 2 demonstrate that SPI-62 can differentially modulate the osteoporotic, compared to the immune suppressive and anti-inflammatory, effects of prednisolone. Cohorts 3 & 4 demonstrated that doubling of the prednisolone dosage given with clofutriben (SPI-62) provides similar efficacy as prednisolone 10 mg, while maintaining the safety advantage over prednisolone by itself.

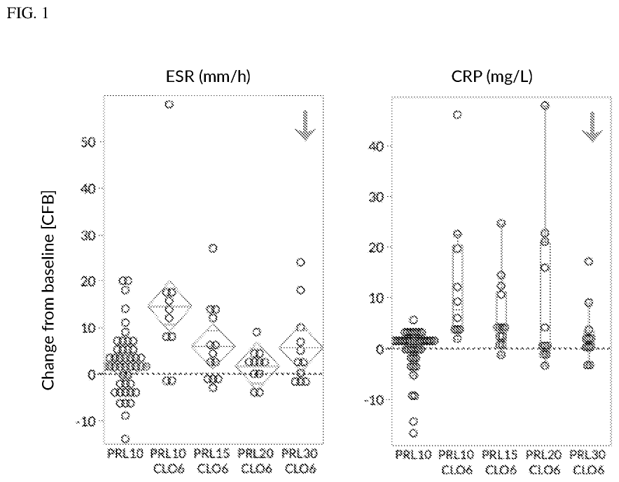

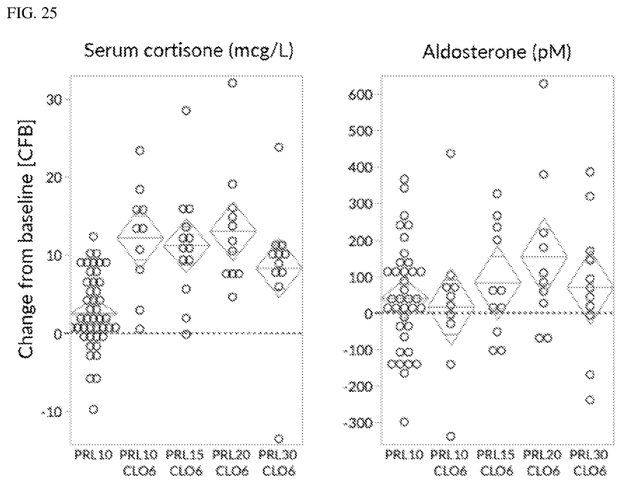

FIGS. 1-26 compare the change from baseline during two weeks of treatment with 10 mg prednisolone and placebo (PRL 10), 10 mg prednisolone and 6 mg clofutriben (PRL10 CLO6), 15 mg prednisolone and 6 mg clofutriben (PRL15 CLO6), 20 mg prednisolone and 6 mg clofutriben (PRL20 CLO6), and 30 mg prednisolone and 6 mg clofutriben (PRL30 CLO6). The central horizontal line of each diamond represents the mean of the observed data. The top and bottom vertices represent the 95% confidence interval of the mean. The top and bottom ‘overlap lines’ would indicate statistically significant difference between groups under assumptions of normal data distribution, equal variances, and lack of covariance with baseline value. As one or more of those assumptions is violated for many of the datasets, statistical significance should not be inferred based on the overlap lines in FIGS. 1-26.

With the PRL10 CLO6 regimen, clofutriben was associated with diminished prednisolone potency to control PMR disease activity and reversed or prevented prednisolone-associated changes on the bone turnover biomarkers osteocalcin (OC), prothrombin type 1 N-propeptide (P1NP), and C-terminal collagen crosslinks (CTX). With the PRL15 CLO6 regimen, SPI-62 was still associated with diminished prednisolone potency to control PMR disease activity but less so compared to the PRL10 CLO6 regimen. Favorable changes on OC and P1NP were observed, as well as favorable changes to cholesterol, triglycerides, APTT, heart rate, and morning serum cortisol. The PRL20 CLO6 and PRL30 CLO6 regimens demonstrated that a prednisolone dose adjustment of 2× when co-administered with clofutriben was sufficient to stabilize how patients felt and functioned; dosages of 1.5× or less were determined to be insufficient to provide the needed efficacy. The safety advantages conferred by clofutriben co-administration were maintained.

Assessment of relative PMR disease control was based on four criteria.

Criterion 1—Relapse with Adverse events or PMR aggravated, i.e., when the investigator considers that the patient's condition indicates clinically meaningful exacerbation of their PMR. The incidence of adverse events of PMR aggravated was 0/50 for prednisolone 10 mg with placebo for SPI-62, 7/13 for prednisolone 10 mg with SPI-62 6 mg, and 1/12 for prednisolone 15 mg with SPI-62 6 mg. A 50% adjustment of prednisolone dose restored the relapse rate to like that without SPI-62. The incidence was 2/11 for PRL20 CLO6 and 1/11 for PRL30 CLO6. Critically, of the five groups 0/50 (PRL10), 5/13 (PRL10 CLO6), 0/12 (PRL15 CLO6), 0/11 (PRL20 CLO6), and 0/11 (PRL30 CLO6) showed PMR relapse, i.e., an increase of PMR symptoms or disease activity of sufficient magnitude that a prednisolone dose increase was indicated.

Criterion 2—Acute phase markers C-reactive protein (CRP) and erythrocyte sedimentation rate (ESR), which are standard parameters clinicians use to quantitate systemic inflammation in PMR. These are specified as measurements of the core domain of systemic inflammation by the Outcomes Measures in Rheumatology Clinical Trials (OMERACT) PMR Working Group (PMR WG). CRP and ESR showed clinically meaningful increases in participants who received prednisolone 10 mg with SPI-62 and increases of smaller (but still clinically meaningful) magnitude in participants who received prednisolone 15 mg with SPI-62. Increasing the dosages of prednisolone to 20 and 30 mg showed an even smaller increase in participants (FIG. 1).

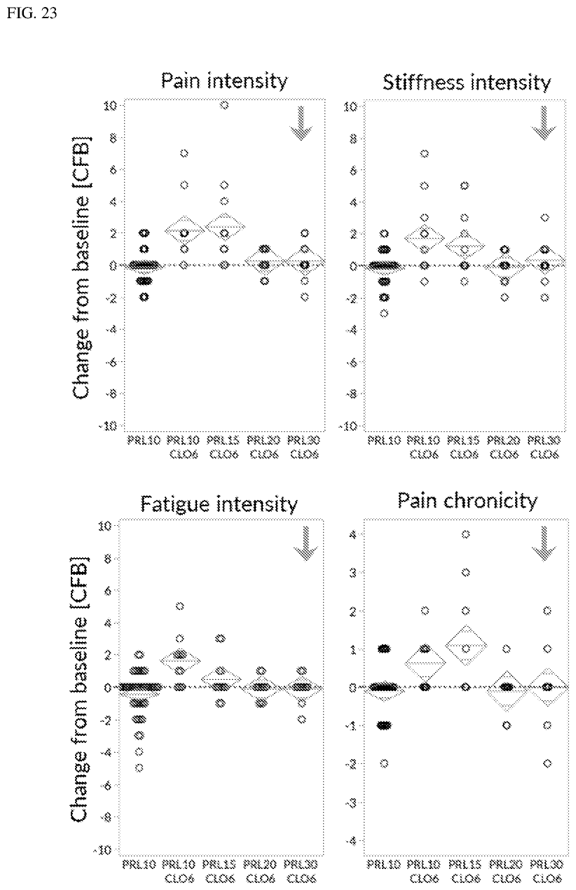

Criterion 3—OMERACT PMR WG specified pain, stiffness, and fatigue intensity, and pain chronicity, as the core domains of how patients feel. OMERACT PMR WG consensus numeric rating scales (NRS) for maximum intensity (0-10, anchored with ‘no [symptom]’ for 0 and ‘very severe [symptom]’ for 10) and chronicity (0-4, anchored with descriptive terms for proportion of time) with 24-hour look back period were completed daily by trial participants. Participants endorsed increased maximum pain, stiffness, and fatigue intensity, and pain chronicity, when clofutriben 6 mg was co-administered with prednisolone 10 or 15 mg, but not when clofutriben was co-administered with prednisolone 20 or 30 mg, indicating that doubling of the dosage stabilized how patients felt similar to how they felt when treated with prednisolone by itself (FIG. 23). Participants completed NRS with a 7-day look back period during each trial visit. Some consider such instruments less sensitive as patients do not recall symptoms over 7 days as accurately. However, the results were similar to those reported on daily symptom instruments. Participants endorsed increased symptoms during treatment with clofutriben 6 mg+prednisolone 10 or 15 mg, but less so during treatment with clofutriben 6 mg+prednisolone 20 or 30 mg.

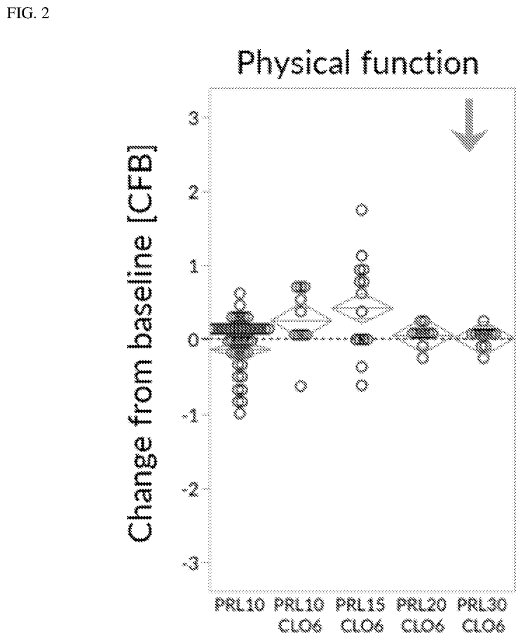

Criterion 4—Health Assessment Questionnaire Disability Index (HAQ-DI) is widely used to assess difficulty with IADLs in rheumatology clinical trials. OMERACT PMR WG specified physical function (i.e., difficulty with IADLs) as the core domain of how patients function. Participants endorsed increased difficulty with IADLs during treatment with clofutriben 6 mg+prednisolone 10 or 15 mg. While the mean change was not different across the 10 and 15 mg regimens, increasing the dosages of prednisolone to 20 and 30 mg notably decreased patient reports of patient difficulty with IADLs relative to the increases observed with lower prednisolone doses, indicating a doubling of the dosage stabilized how patients functioned similar to that of prednisolone by itself (FIG. 2).

Exploratory molecular and cellular biomarkers with potential relationship to PMR disease activity based on results in the literature were selected to provide granular insight into how prednisolone and SPI-62 influence the immune system in patients with PMR.

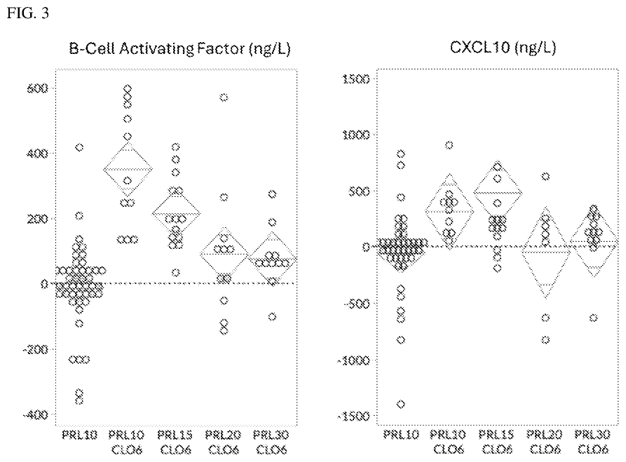

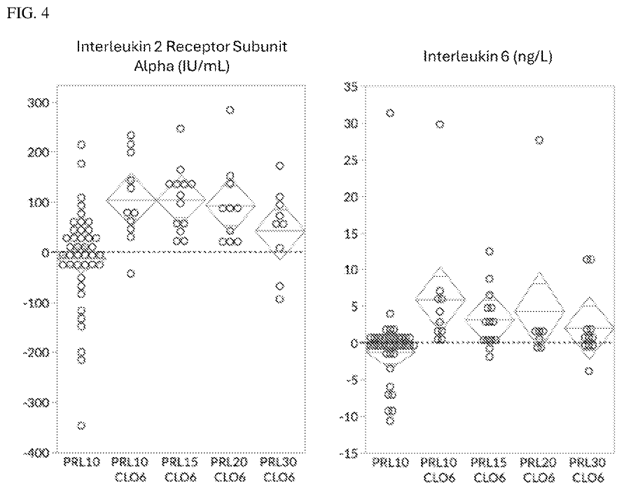

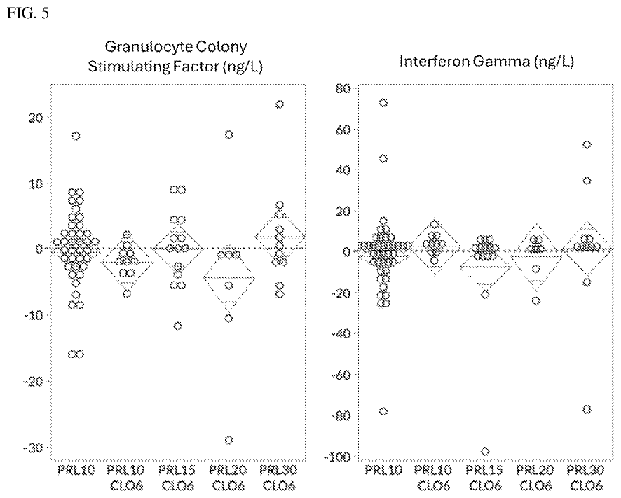

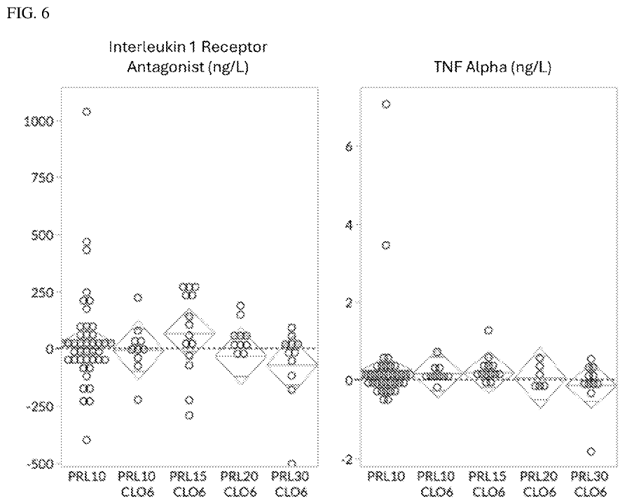

A 13-plex cytokine panel showed three patterns:
Granulocyte-macrophage colony stimulating factor (GM-CSF), interleukin (IL)-1β, IL-2, IL-10, and IL-17 were uninformative as all or most data were below the limit of quantitation.
B cell activating factor (BAFF), CXC motif chemokine ligand 10 (CXCL10), IL-2 receptor subunit A (IL-2RA), and IL-6 were sensitive to SPI-62. Levels increased in participants who received prednisolone 10 mg with SPI-62 6 mg and increased less in participants who received prednisolone 15 mg with SPI-62 6 mg. Smaller increases in BAFF and CXCL10 when compared to placebo were observed in the 20 and 30 mg participants (FIGS. 3 and 4).
Granulocyte colony stimulating factor (G-CSF), IL-1 receptor antagonist (IL-1RA), and interferon gamma (IFNγ) did not show sensitivity to SPI-62. These observations highlight that some well-known immune pathways known to be suppressed by glucocorticoids (e.g., IFNγ) might not be influenced meaningfully by HSD-1 inhibition or might be less relevant in PMR (FIGS. 5 and 6).

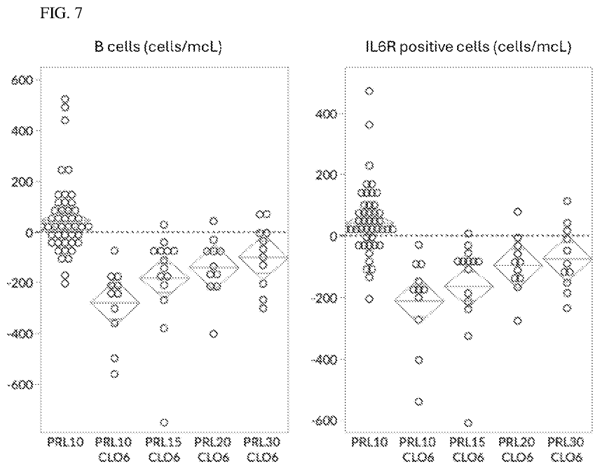

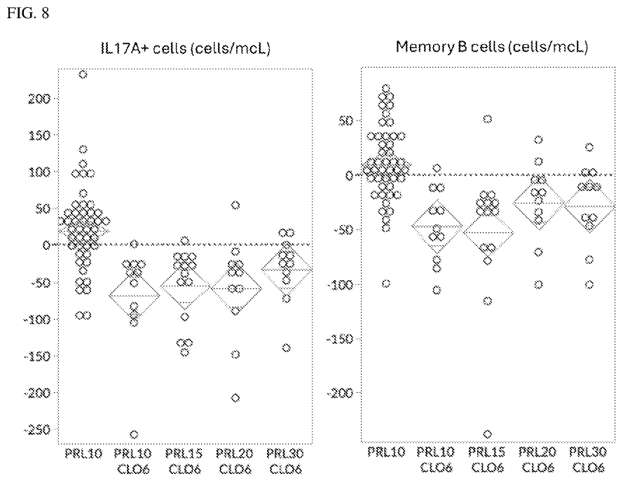

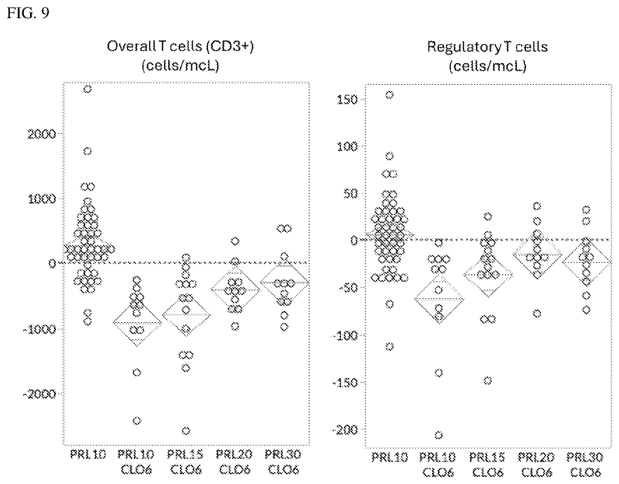

Counts of several circulating lymphoid cell types were determined by quantitative PCR for lineage-specific DNA methylation patterns. SPI-62 was associated with decreases of all counted lineages, which were not fully ameliorated by prednisolone dose adjustment (FIGS. 7-9). These decreases may represent reversal of prednisolone-associated suppression of cellular extravasation to tissue sites of inflammation, and these patterns might reflect movement, rather than depletion, of cells. B and IL-6R+ cells (FIG. 7) are representative examples of the patterns seen for memory B, CD3+T, Th17, and Treg cells (FIGS. 8-9).

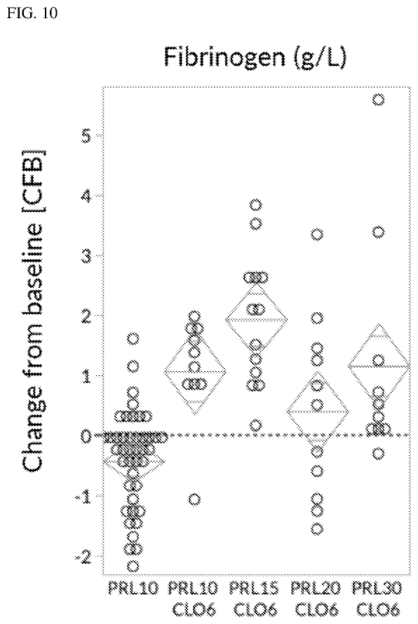

Fibrinogen (PF) is an acute phase marker of general inflammation that, in two studies, showed superior discriminative value between well- and poorly-controlled PMR than did CRP or ESR. However, the PF assay requires very fresh or frozen samples which renders it impractical in many clinical settings. PF levels increased with SPI-62, with a numerically larger increase at the PRL15 CLO6 regimen. PF levels decreased somewhat with increased prednisolone dosages of 20 and 30 mg, though inconsistently (FIG. 10).

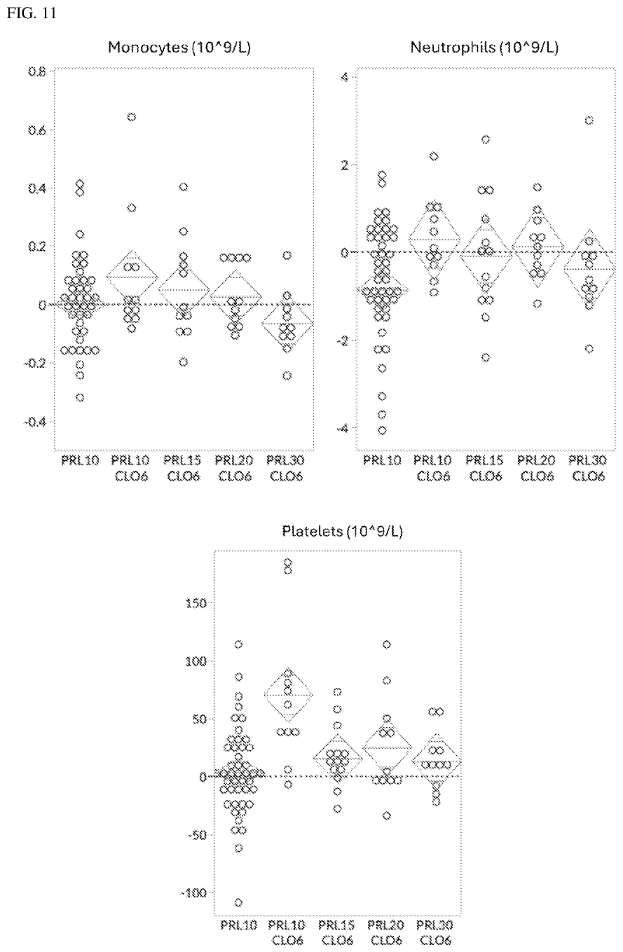

Monocyte, neutrophil, and platelet counts have been observed to correlate with disease activity in PMR. SPI-62 was initially associated with anticipated increases in each parameter. SPI-62-associated increases were numerically smaller for monocytes and neutrophils, and reversed completely for platelets, with a 50% prednisolone dose adjustment (FIG. 11). This reversal was further observed at the 20 and 30 mg prednisolone dosing regimens.

### Assessment of Prednisolone Toxicity

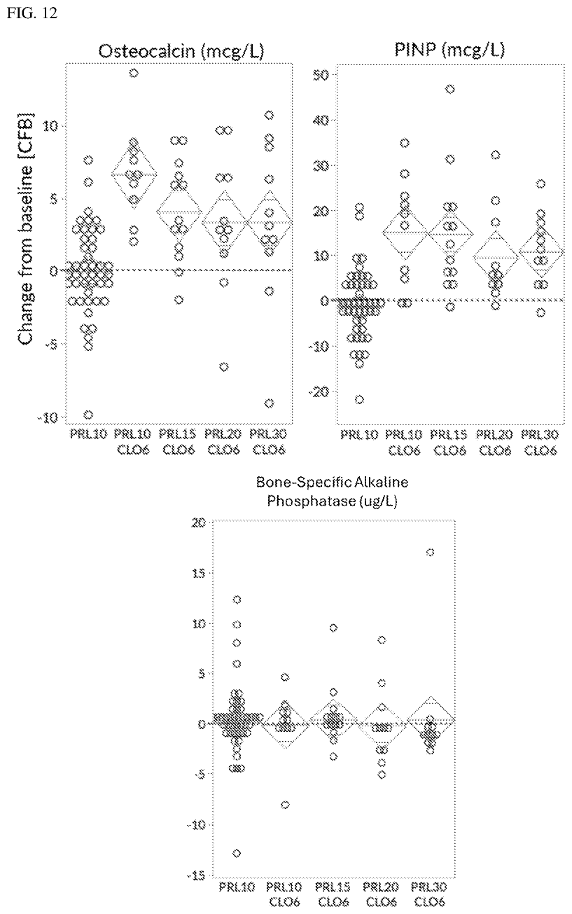

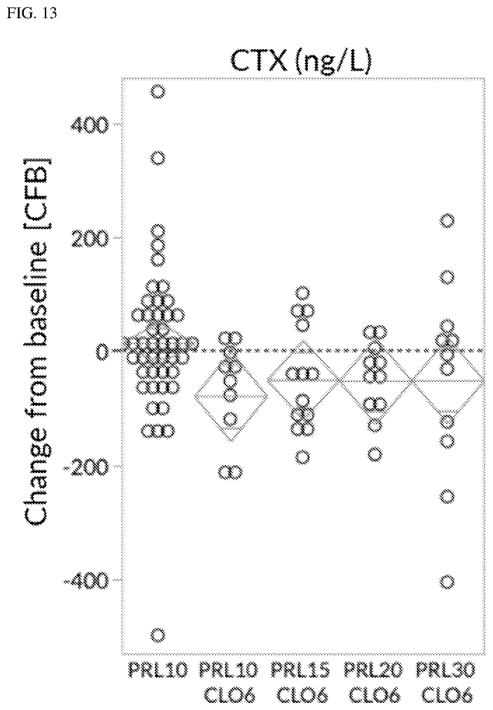

Assessment of prednisolone toxicity focused first on bone turnover biomarkers, which were expected to be the most sensitive based on published evidence of substantial changes within a few days of glucocorticoid administration that correlated with hepatic HSD-1 activity. SPI-62 was associated with changes consistent with an anti-GIOP phenotype, i.e., increases of the bone formation biomarkers OC and P1NP (FIG. 12), and decreases of the bone resorption biomarker CTX (FIG. 13). BSAP showed low sensitivity (FIG. 12).

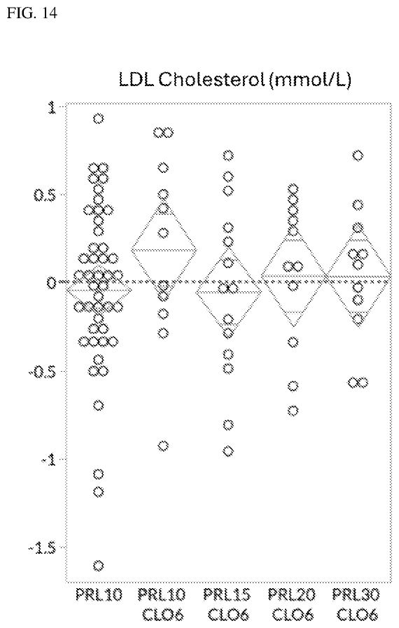

Results in the literature show that LDL cholesterol and triglycerides are not particularly sensitive to short-term GC administration, for example dexamethasone 3 mg (equivalent to prednisolone 20 mg) administered to healthy adults for 5 days. The confidence intervals of mean change of cholesterol, LDL cholesterol, and triglycerides in participants who received prednisolone 10 mg with placebo for SPI-62 for 2 weeks each encompassed zero. Those parameters were initially considered to not be sensitive to SPI-62 co-administration based on data from the 10 and 15 mg prednisolone groups, though with additional data it became apparent that triglycerides and cholesterol were sensitive even at these levels (FIGS. 14, 19, and 20).

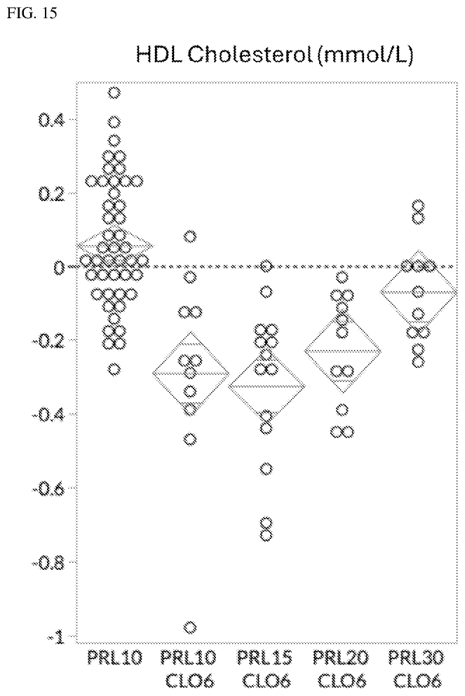

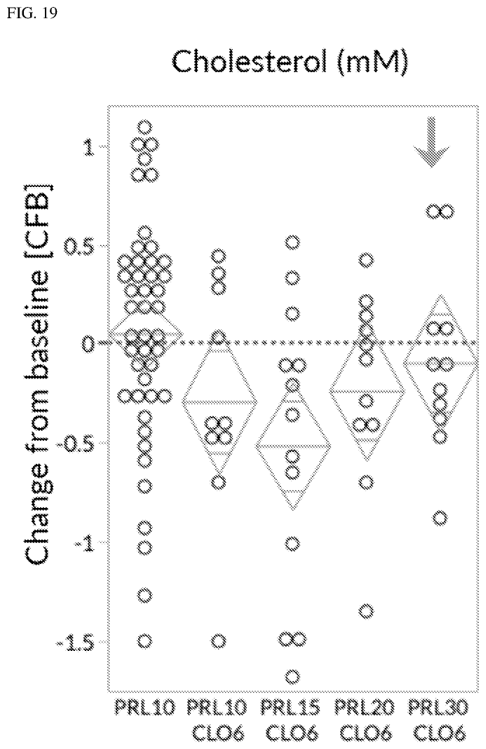

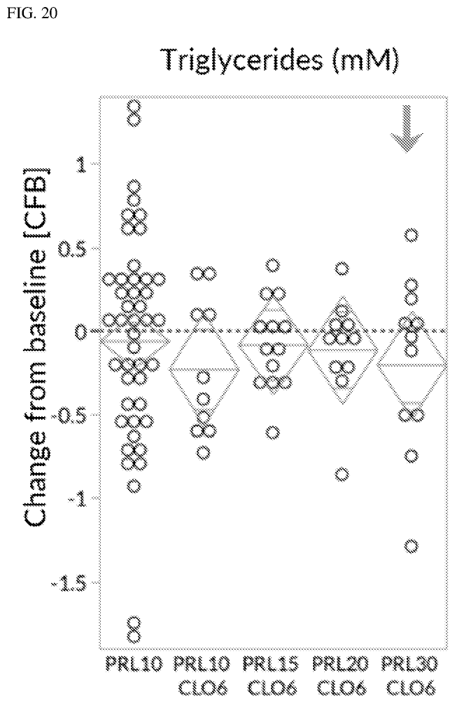

In contrast, that dexamethasone regimen was associated with a clinically meaningful increase of HDL cholesterol, an observation that falls neatly into neither prednisolone efficacy nor toxicity. SPI-62 6 mg co-administered for 2 weeks with prednisolone 10 or 15 mg was associated with a decrease of HDL cholesterol almost exactly equal in magnitude to the increase associated with dexamethasone in healthy adults (FIG. 15). Whether GC-associated HDL increase should be considered GC toxicity is not clear. Additional testing of clofutriben co-administration with 20 and 30 mg of prednisolone was subsequently shown to further improve both cholesterol (FIG. 19) and triglycerides (FIG. 20) with increases in the dosages of prednisolone. With additional data, favorable changes in the earlier data are now evident as part of a dose-response relationship whereas earlier they were not clearly distinct. About half of the participants (represented by colored circles) were on cholesterol-lowering agents, while no participants received any additional triglyceride lowering agents. The improvements in cholesterol were attributed mainly to a decrease in HDL, representing a reversal of the HDL-increasing effect of GC by clofutriben.

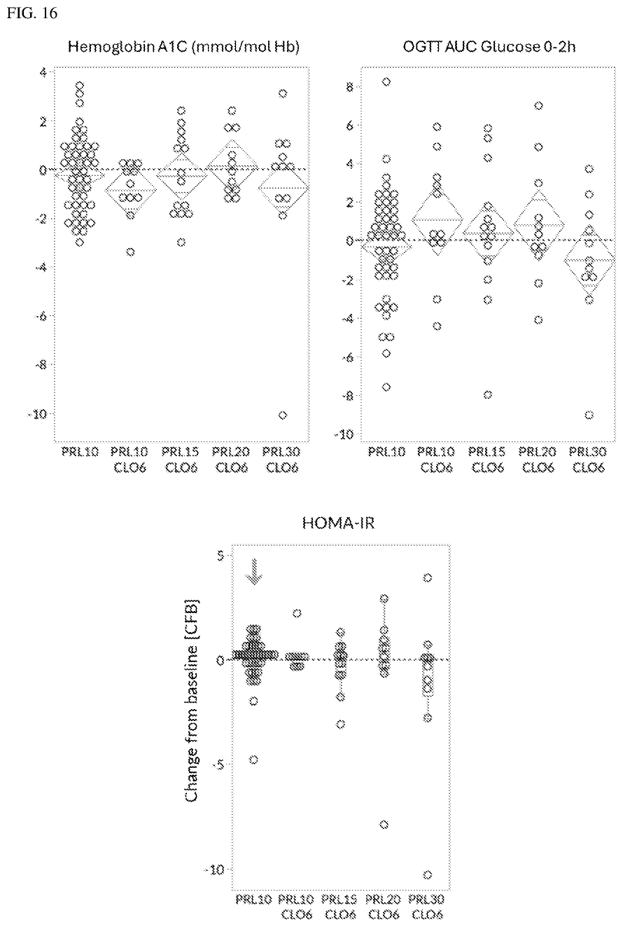

Results in the literature show that OGTT glucose AUC and HOMA-IR (homeostatic model assessment of insulin resistance), common measures of post-prandial and basal glycemic control, were less sensitive to prednisone (prodrug of prednisolone, hence dose equivalent) 10 mg administered to healthy adults for 7 days, compared to prednisone 25 or 60 mg. Those parameters and HbA1c (an integrated measure of overall glycemic control generally sensitive only over longer assessment periods) were not sensitive to SPI-62 co-administration. The small increases on OGTT glucose AUC are similar in magnitude to those that have been observed in healthy adults treated with placebo and are not clinically meaningful. The HbA1c decrease associated with 10 mg prednisolone+placebo for SPI-62 was not expected and was not replicated. The mean changes associated with SPI-62 co-administration were close to zero (FIG. 16). In prior trials, SPI-62 (without prednisolone) was associated with a clinically meaningful decrease of HbA1c at 6 weeks in patients with diabetic peripheral neuropathy.

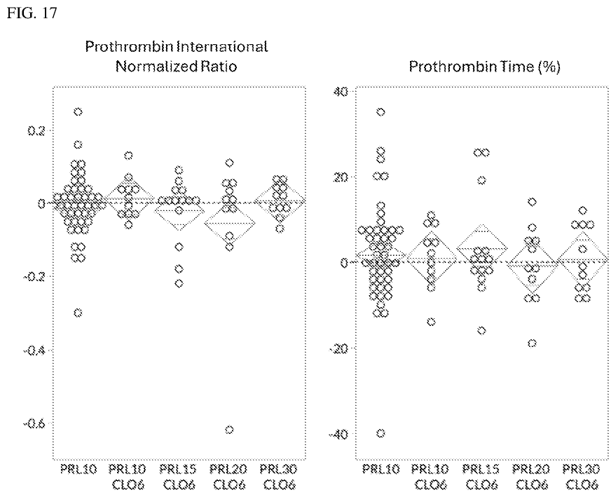

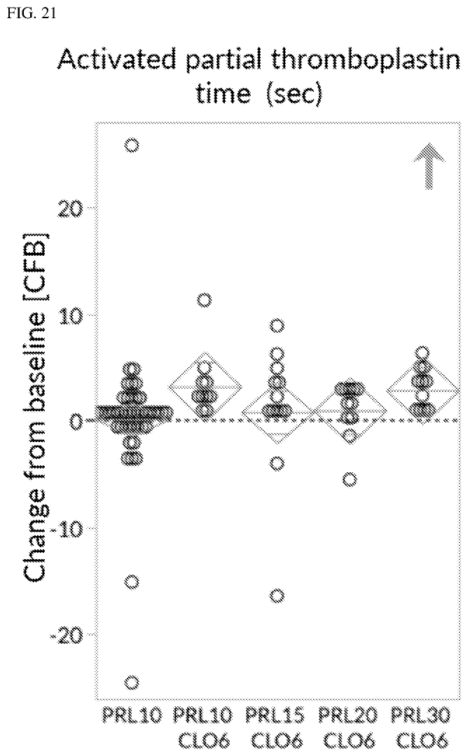

Coagulation parameters INR and PT were not observed to be sensitive to SPI-62 co-administration with prednisolone at any prednisolone dose (FIG. 17). Clofutriben was observed to improve activated partial thromboplastin time (APTT), in keeping with a recent report that found APTT more sensitive to high doses of GC (FIG. 21). APTT below the reference range could indicate prothrombotic risk.

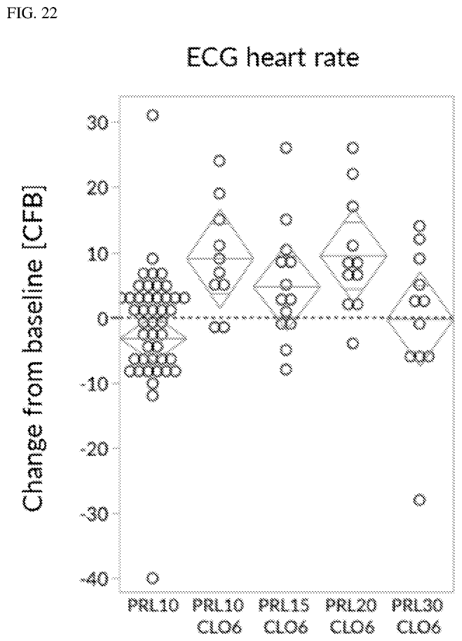

Results in the literature show that dexamethasone 3 mg (equivalent to prednisolone 20 mg) administered to healthy adults for 5 days was associated with an ECG heart rate decrease of approximately 12 bpm. SPI-62 6 mg co-administered for 2 weeks with prednisolone 10 or 15 mg was associated with ECG heart rate increase of approximately 9 or 5 bpm, which might represent reversal of a prednisolone effect. No effect on ECG heart rate was associated with SPI-62 in prior trials, in which participants did not receive GCs. Additional testing of higher doses of 20 co-administered with clofutriben found that the effect on ECG heart rate was maintained, though no longer observed at a 30 mg prednisolone dose (FIG. 22).

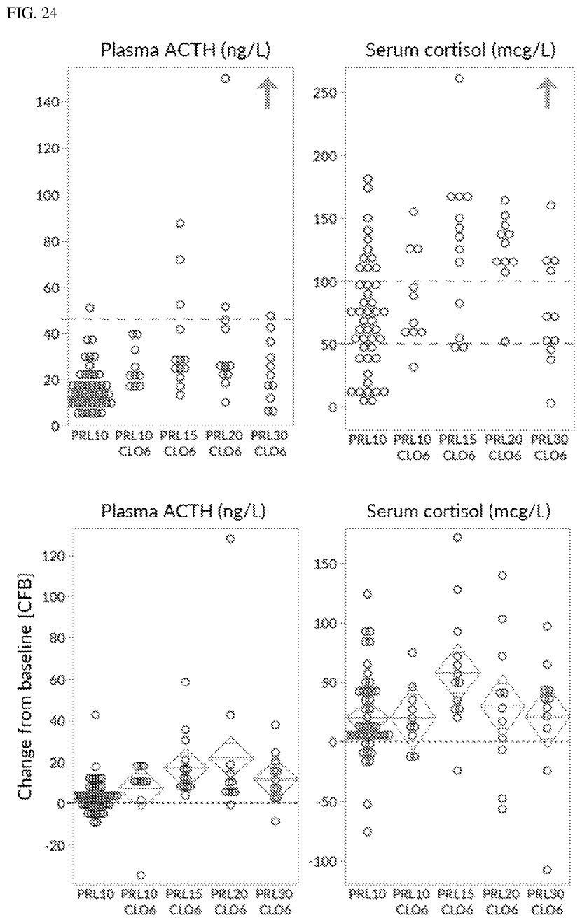

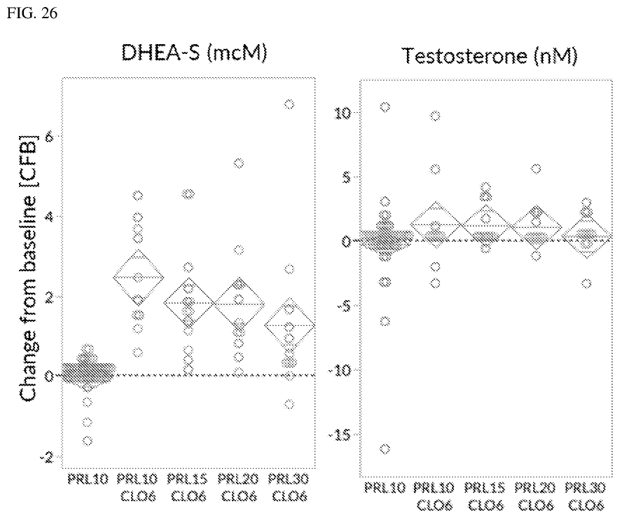

Clofutriben was found to be associated with hypothalamic-pituitary-adrenal (HPA) axis de-repression in patients that received prednisolone. Both plasma ACTH and serum cortisol was found to increase in patients receiving clofutriben compared to those on placebo (FIG. 24). The cortisol increase may indicate a reduced risk for adrenal insufficiency or glucocorticoid withdrawal syndrome, while the ACTH increase is associated with increased androgens. Accordingly, DHEA-S and testosterone were found to increase, within or close to the reference range, relative to placebo (FIG. 26).

### Assessment of SPI-62 Pharmacological Activity

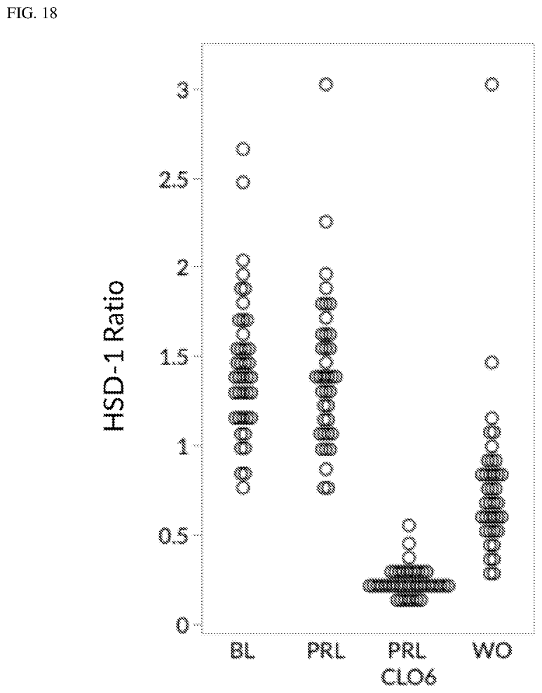

Data from all cohorts were analyzed together to confirm the pharmacological effect of SPI-62 on hepatic HSD-1 activity. In hepatocytes, 5- and 3-steroid reductases, the latter a liver-specific enzyme, convert cortisol and cortisone to tetrahydro metabolites which are excreted in urine. The urinary ratio (tetrahydrocortisol+allotetrahydrocortisol)/tetrahydrocortisone is an accepted biomarker of hepatic HSD-1 activity. Morning spot urine samples were collected at Baseline (BL), after 2 weeks of prednisolone 10 mg treatment (PRL), after 2 weeks of prednisolone 10-30 mg+clofutriben 6 mg treatment (PRL CLO6), and after 4 weeks washout following the last clofutriben dose (WO). Because some cortisol distributes into hepatocytes from free (not protein bound) cortisol in circulation, the HSD-1 ratio does not go to zero with full HSD-1 inhibition. A mean value of approximately 0.2 or lower represents full hepatic HSD-1 inhibition which was achieved with SPI-62 in this trial. The similarity between BL and PBO is an example of the repeatability of the HSD-1 ratio in spot urine samples. Substantial hepatic HSD-1 inhibition was observed 4 weeks after the last dose of SPI-62 (FIG. 18).

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications, and publications to provide yet further embodiments.

These and other changes can be made to the embodiments in light of the above-detailed description. In general, in the following claims, the terms used should not be construed to limit the claims to the specific embodiments disclosed in the specification and the claims but should be construed to include all possible embodiments along with the full scope of equivalents to which such claims are entitled. Accordingly, the claims are not limited by the disclosure.

What is claimed is:

1. A fixed dose combination comprising no more than about 6 mg of clofutriben and between about 1.5 and about 150 mg of prednisolone.

2. The fixed dose combination of claim 1, comprising no more than about 3 mg of clofutriben and between about 1.5 and about 7.5 mg of prednisolone.

3. The fixed dose combination of claim 2, comprising about 1.2 mg of clofutriben.

4. The fixed dose combination of claim 3, comprising between about 2 and about 3 mg of prednisolone.

5. The fixed dose combination of claim 4, wherein the fixed dose combination is sufficient to achieve a therapeutic effect corresponding to a dose of about 1 mg of prednisolone administered alone.

6. The fixed dose combination of claim 2, comprising about 3 mg of clofutriben.

7. The fixed dose combination of claim 6, comprising between about 5 and about 7.5 mg of prednisolone.

8. The fixed dose combination of claim 7, wherein the fixed dose combination is sufficient to achieve a therapeutic effect corresponding to a dose of about 2.5 mg of prednisolone administered alone.

9. The fixed dose combination of claim 1, comprising about 6 mg of clofutriben and between about 5 and about 150 mg of prednisolone.

10. The fixed dose combination of claim 5, comprising between about 10 and about 15 mg of prednisolone.

11. The fixed dose combination of claim 10, wherein the fixed dose combination is sufficient to achieve a therapeutic effect corresponding to a dose of about 5 mg of prednisolone administered alone.

12. The fixed dose combination of claim 5, comprising between about 20 and about 30 mg of prednisolone.

13. The fixed dose combination of claim 12, wherein the combined dosage is sufficient to achieve a therapeutic effect corresponding to a dose of about 10 mg of prednisolone administered alone.

14. The fixed dose combination of claim 5, comprising between about 40 and about 60 mg of prednisolone.

15. The fixed dose combination of claim 14, wherein the combined dosage is sufficient to achieve a therapeutic effect corresponding to a dose of about 20 mg of prednisolone administered alone.

16. The fixed dose combination of claim 5, comprising between about 100 and about 150 mg of prednisolone.

17. The fixed dose combination of claim 16, wherein the combined dosage is sufficient to achieve a therapeutic effect corresponding to a dose of about 50 mg of prednisolone administered alone.

18. The fixed dose combination of claim 1, wherein the clofutriben and the prednisolone are administered separately.

19. The fixed dose combination of claim 1, wherein the clofutriben and the prednisolone are co-formulated.

20. The fixed dose combination of claim 1, wherein the clofutriben and prednisolone are both formulated for immediate release.

21. The fixed dose combination of claim 1, wherein the fixed dose combination exhibits lowered toxicity than prednisolone administered alone.

22. The fixed dose combination of claim 21, wherein the toxicity is glucocorticoid-induced osteoporosis (GIOP) and wherein the lowered toxicity is characterized by increased osteocalcin, increased procollagen type 1 N-terminal propeptide, increased bone-specific alkaline phosphatase, or decreased C-terminal telopeptide of type 1 collagen.

23. The fixed dose combination of claim 21, wherein the toxicity is bradycardia and wherein the lowered toxicity is characterized by increased heart rate.

24. A method of treatment of a prednisolone-mediated disease, comprising administering a fixed dose combination of claim 1 to a patient in need thereof.

25. The method of claim 24, wherein the prednisolone-mediated disease is chosen from an autoimmune disease, an inflammatory condition, an allergic condition, a dermatologic disease, an endocrine condition, a gastrointestinal disease, a hematologic disease, a neoplastic condition, a nervous system condition, an ophthalmic condition, a pulmonary disease, a renal condition, a rheumatologic condition, and an infectious disease.

26. The method of claim 25, wherein the autoimmune disease is polymyalgia rheumatica.

27. The method of claim 25, wherein the prednisolone-mediated disease is chosen from:
(a) an allergic condition chosen from atopic dermatitis, drug hypersensitivity, seasonal or perennial allergic rhinitis, and serum sickness;
(b) a dermatologic disease chosen from bullous dermatitis herpetiformis, contact dermatitis, exfoliative erythroderma, mycosis fungoides, pemphigus, and severe erythema multiforme (Stevens-Johnson syndrome);
(c) an endocrine condition chosen from congenital adrenal hyperplasia, hypercalcemia of malignancy, nonsuppurative thyroiditis, and primary or secondary adrenocortical insufficiency;
(d) a gastrointestinal disease chosen from Crohn's disease and ulcerative colitis;
(e) a hematologic disease chosen from acquired (autoimmune) hemolytic anemia, Diamond-Blackfan anemia, adult idiopathic thrombocytopenic purpura, pure red cell aplasia, and adult secondary thrombocytopenia;
(f) a neoplastic condition chosen from acute leukemia and aggressive lymphomas;
(g) a nervous system condition chosen from multiple sclerosis and cerebral edema associated with a primary or metastatic brain tumor, craniotomy, or head injury;
(h) an ophthalmic condition chosen from sympathetic ophthalmia, uveitis, and ocular inflammatory conditions unresponsive to topical corticosteroids;
(i) a pulmonary disease chosen from chronic obstructive pulmonary disease (COPD), allergic bronchopulmonary aspergillosis, aspiration pneumonitis, asthma, fulminating or disseminated pulmonary tuberculosis, hypersensitivity pneumonitis, idiopathic bronchiolitis obliterans, idiopathic eosinophilic pneumonias, idiopathic pulmonary fibrosis, Pneumocystis carinii pneumonia (PCP), and symptomatic sarcoidosis;
(j) a renal condition chosen from nephrotic syndrome;
(k) a rheumatologic condition chosen from acute gouty arthritis, ankylosing spondylitis, dermatomyositis, polymyositis, polymyalgia rheumatica, temporal arteritis, psoriatic arthritis, relapsing polychondritis, rheumatoid arthritis, juvenile rheumatoid arthritis, Sjogren's syndrome, systemic lupus erythematosus, and vasculitis; and
(l) an infectious disease chosen from trichinosis and tuberculous meningitis.

28. The method of claim 24, wherein the administration results in an increase in bone formation or a decrease in bone resorption in the patient.

29. The method of claim 28, wherein the increased bone formation is characterized by increased osteocalcin, increased procollagen type 1 N-terminal propeptide, increased bone-specific alkaline phosphatase, or the decreased bone resorption is characterized by decreased C-terminal telopeptide of type 1 collagen.
